(19) United States
(12) Patent Application Publication
(10) Pub. No.: US 2022/0280314 A1
(43) Pub. Date: Sep. 8, 2022

(54) LOCKING STRUCTURE OF KNEE JOINT

(71) Applicant: Ken Dall Enterprise Co., Ltd., New Taipei City (TW)

(72) Inventors: Chia-Pao Cheng, New Taipei City (TW); Chih-Hsuan Liang, New Taipei City (TW)

(21) Appl. No.: 17/195,580

(22) Filed: Mar. 8, 2021

(51) Int. Cl. A61F 2/64

(52) U.S. Cl. CPC A61F 2/64; A61F 2002/5072

(57) ABSTRACT

An improvement on a locking structure of a knee joint is provided. The knee joint includes a joint upper seat, a joint lower seat, and a fastening clamp including a rotary axle connected to the joint upper seat and a fastening clamp through-hole connected to the joint lower seat. The fastening clamp includes a fastening clamp channel. The rotary axle includes an axle opening. The fastening clamp includes a fastening groove in which a fixer connection rod is pivotally connected. The fixer connection rod has two ends respectively connected to a fixer disposed in the fastening clamp channel and a connecting rod to which a pull cable is coupled. The connecting rod is rotated by the pull cable to selectively cause the fixer to move in opposite directions in the fastening clamp channel to set the knee joint in a rotatable state and a fixed state, respectively.

### TECHNICAL FIELD OF THE INVENTION

The present invention relates to an improvement on a locking structure of a knee joint.

### DESCRIPTION OF THE PRIOR ART

A known knee joint is generally made up of a knee upper portion, a knee lower part, and a coupling element connected therebetween. The knee upper part rotatably arranged about a rotational axle of the coupling element. The rotational axle may compress an elastic element of the knee lower part to provide the knee joint that is bent and enters a knee stretching posture with a knee-stretching pushing force in order to ensure security of walking for a user.

Improvements have been made later, such as U.S. Pat. No. 10,231,850, which proposes a prosthetic joint, of which a structure, according to the disclosure of the document, includes a fixing device that is detachable and is arranged between the knee upper part and the coupling element to fix at least one rotational position. Such a fixing device is a pawl hinged to the knee upper part and is engageable into a recess formed in the coupling element. One can appreciated, based on the structure discussed above, switching can be made between a locked state and a released state by adjusting the pawl relative to the recess so that a user may use a simple structure to easily perform fixing of a bending position.

However, there are several problems of the above-described structure to be overcome, which are summarized as follows:

(1) The fixing device arranged between the knee upper part and the coupling element is partially exposed to the outside of the prosthetic joint, making the operation of the structure easily interfered with by external factors.

(2) Switching between the locked state and the released state is generally realized through rotating an angle of the knee joint to achieve fixing, and when the state switching of the knee joint is carried out very quickly, an impact force generated thereby would directly affect the stability of the fixing device, leading to discomfort of the prosthetic joint wearer.

In summary, the known structure for locking switching applied to a knee joint, although operable to lock or release the knee joint, is not perfect and an improvement on such a structure is necessary.

### SUMMARY OF THE INVENTION

To achieve the above purpose, the present invention provides an improvement on a locking structure of a knee joint, comprising: a joint upper seat, which is extended downward to form a pair of joint upper seat through-holes; a joint lower seat, which comprises an elastic body, the joint lower seat being extended upward to form a pair of joint lower seat through-holes; a fastening clamp, which is provided therein with a fastening clamp through-hole and a rotary axle, the rotary axle corresponding to and connected to the joint upper seat by a pair of mounting bolts, the fastening clamp through-hole corresponding to and connected to the joint lower seat through-holes by a connection shaft penetrating therethrough.

A fastening clamp channel is formed in the fastening clamp and is extended to and in communication with the rotary axle. The rotary axle is formed with an axle opening. The fastening clamp is extended from an outside thereof to form a fastening clamp groove. The fastening clamp groove is collaborative with a positioning piece penetrating therethrough to pivotally connect a fixer connection rod in the fastening clamp groove. The fixer connection rod has an end, which is collaborative with an axle penetrating therethrough to penetrate through a fixer to set the fixer in the fastening clamp channel. The fixer connecting rod has an opposite end that is collaborative with a bolt to fix to a connecting rod. The connecting rod is coupled to a pull cable. When the connecting rod is pulled upward by the pull cable to rotate, the fixer moves downward in the fastening clamp channel to contact and push the elastic body so as to set the knee joint in a rotatable condition, and when the pull cable stops pulling the connecting rod, the fixer moves upward in the fastening clamp channel to reach into the axle opening to be set in a fixed condition, where the knee joint is set in a fixed condition.

A secondary technical purpose is that the elastic body comprises a spring.

Based on the novel and unique design, compared to the prior art, the present invention is such that a fastening clamp is formed therein with a fastening clamp channel, and a fixer connection rod that has one end connected to a fixer is pivotally connected to a fastening clamp groove provided in an outside of the fastening clamp, wherein a rotary axle provided in the fastening clamp is set in communication with the fastening clamp channel, so that when a pull cable is pulled upward, from the outside, the fixer connection rod is synchronously driven to cause the fixer to move in the fastening clamp channel to free the knee upper seat to rotate without being subject to any constraint; and wherein when the pull cable is released, the fixer is set into engagement with an axle opening provided in the outside of the rotary axle to set the knee upper seat in a constrained state. The present invention allows an artificial limb wearer to make switching between a fixed state and a rotatable state and may also effectively reduce an impact force and protect the structure from exposure to the outside in order to ensure security of use.

### BRIEF DESCRIPTION OF THE DRAWINGS

FIG. 1 is a perspective view of the present invention.

FIG. 2 is an exploded view of the present invention.

FIG. 3 is an exploded view of the present invention, taken from a different perspective.

FIG. 4 is a rear view showing the present invention in an unlocked condition.

FIG. 5 is a cross-sectional view of the present invention taken along line A-A of FIG. 4.

FIG. 6 is a schematic view demonstrating operation of an upper joint seat of the present invention shown in FIG. 5.

FIG. 7 is a rear view showing the present invention in a locked condition.

FIG. 8 is a cross-sectional view, taken along line B-B of FIG. 7, showing the present invention in the locked condition.

### DETAILED DESCRIPTION OF THE PREFERRED EMBODIMENT

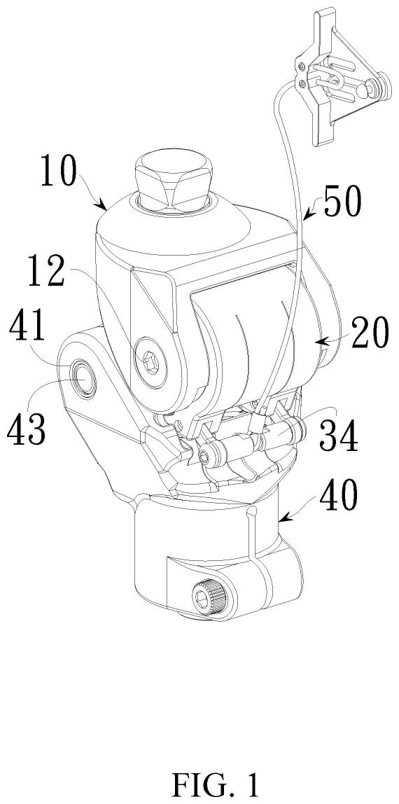

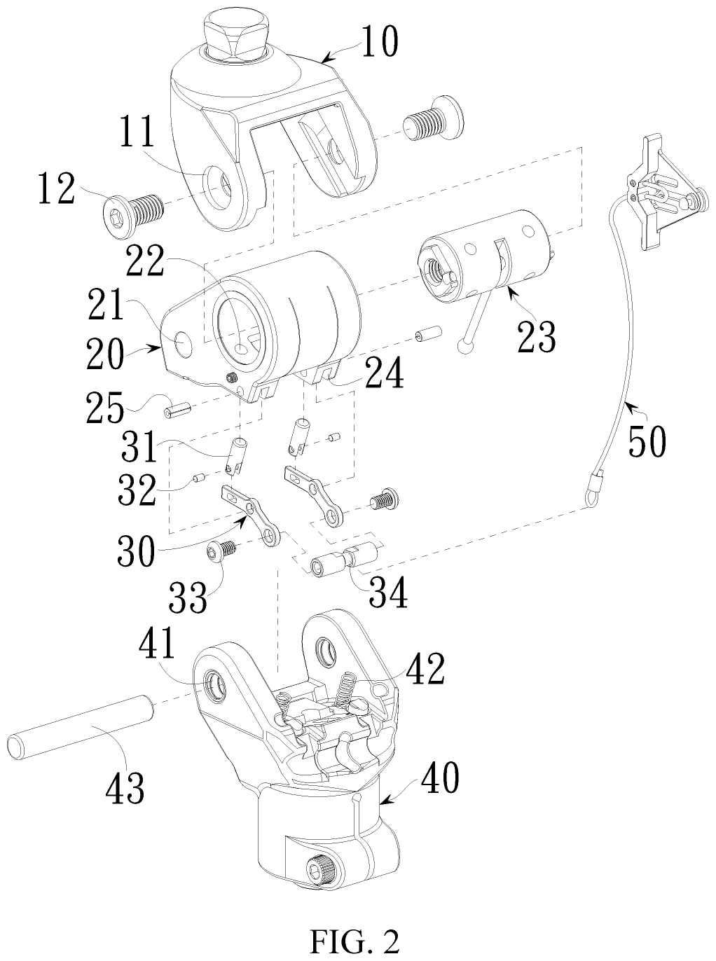

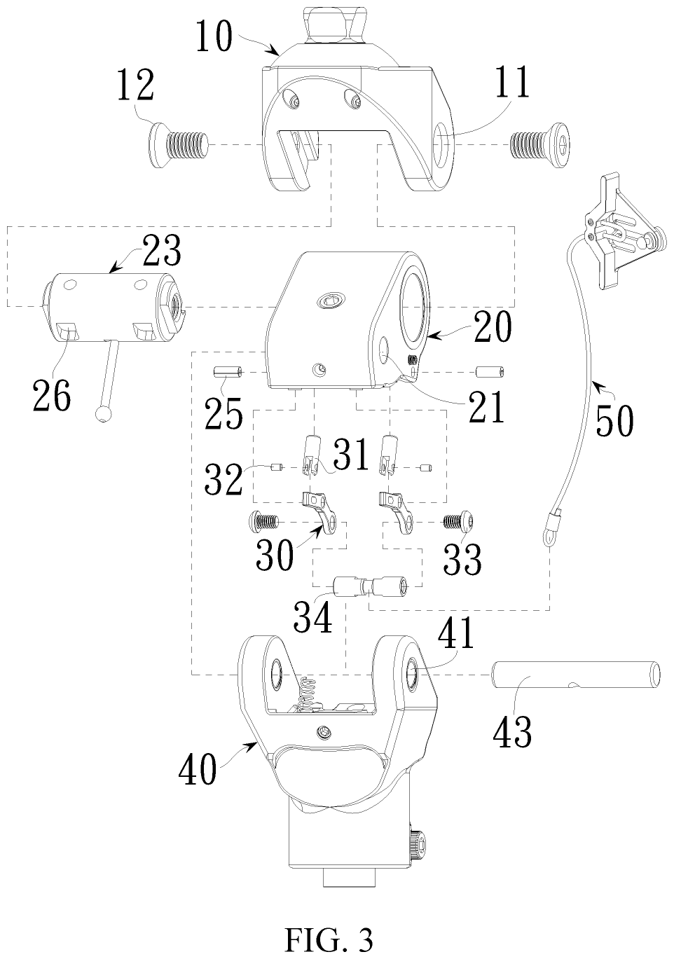

A detailed description, generally according to the present, for a preferred feasible embodiment, is provided hereinafter with reference to FIGS. 1-3, in order to enhance understanding of the present invention. An improvement is provided for a locking structure of a knee joint according to the present invention, in which the knee joint comprises: a joint upper seat (10), which is extended downward to form a pair of joint upper seat through-holes (11); and a joint lower seat (40), which comprises an elastic body (42), wherein the elastic body (42) is embodied as a spring in the context of the present invention and can be replaced with any material exhibiting the same functionality, and is not limited thereto.

The joint lower seat (40) is extended upward to form a pair of joint lower seat through-holes (41). A fastening clamp (20) is provided therein with a fastening clamp through-hole (21) and a rotary axle (23), wherein the rotary axle (23) corresponds to and is connected to the joint upper seat (10) by means of a pair of mounting bolts (12), and the fastening clamp through-hole (21) is set to correspond to and connected to the joint lower seat through-holes (41) by a connection shaft (43) penetrating therethrough.

A fastening clamp channel (22) is formed in the fastening clamp (20) and is extended to and in communication with the rotary axle (23). The rotary axle (23) is also formed with an axle opening (26). A fastening clamp groove (24) is formed and extended in an outside of the fastening clamp (20), or alternatively, the fastening clamp groove (24) is arranged in an inside of the fastening clamp (20), and is not limited thereto. Further, the fastening clamp groove (24) is collaborative with a positioning piece (25) that is set in a penetrating manner to pivotally connect a fixer connection rod (30) in the fastening clamp groove (24). The fixer connection rod (30) has an end, which is collaborative with an axle (32) that is set in a manner of penetrating therethrough to penetrate a fixer (31) to set the fixer (31) in the fastening clamp channel (22), and the fixer connecting rod (30) has an opposite end that is collaborative with a bolt (33) to fix to a connecting rod (34). The connecting rod (34) is coupled to a pull cable (50).

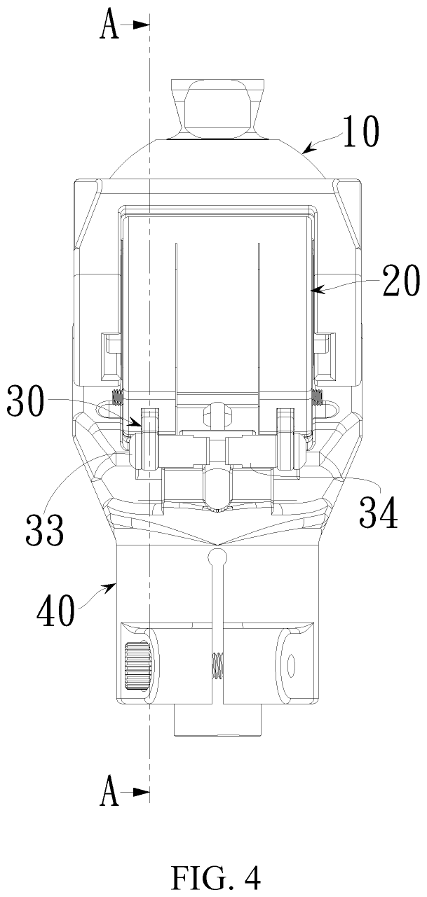

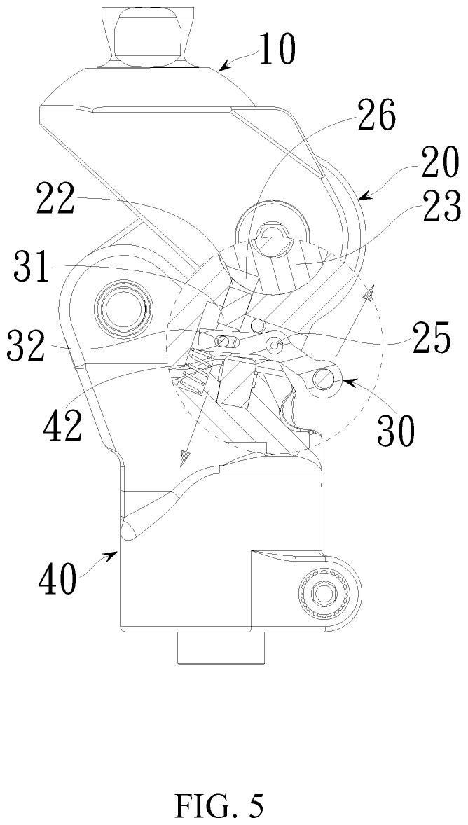

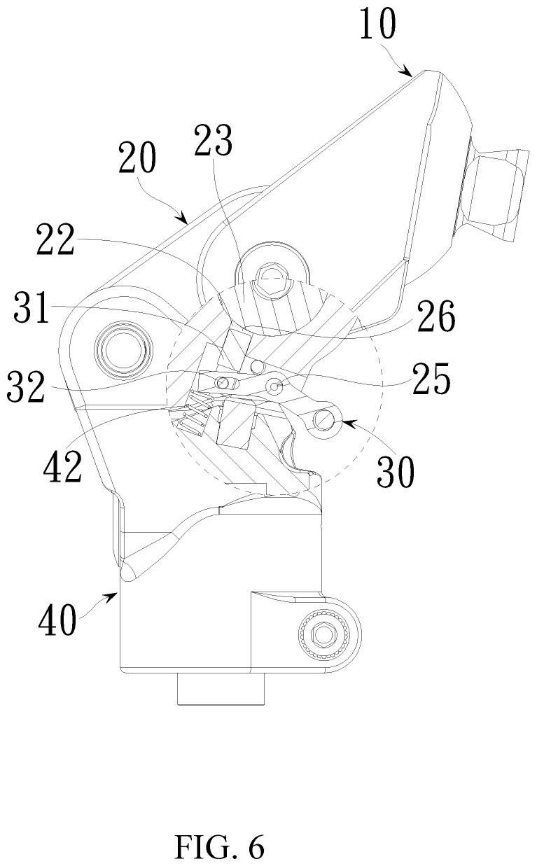

Further referring to FIGS. 4 and 5, when the connecting rod (34) is rotated upward upon being pulled by the pull cable (50), as shown in the cross-section of A-A, the fixer (31) is caused to move downward in the fastening clamp channel (22) to contact and push the elastic body (42), setting the knee joint in a rotatable condition. As shown in FIG. 6, when the knee joint is set in the rotatable condition, the knee upper seat (10) is allowed to perform a rotating motion.

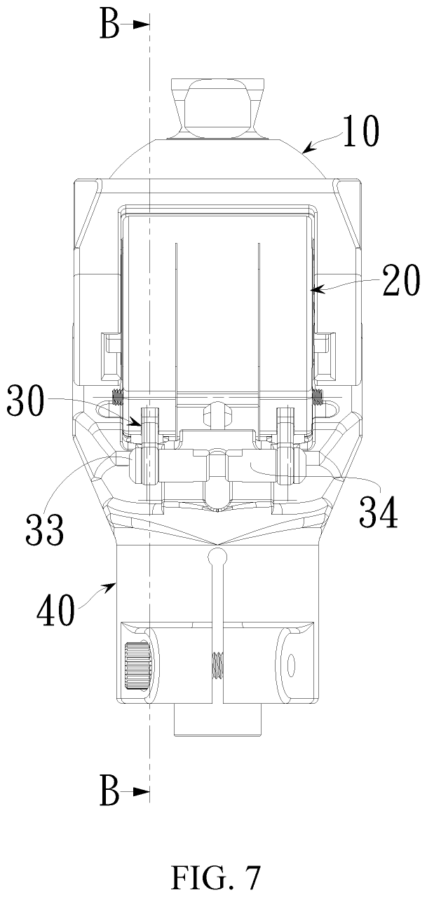

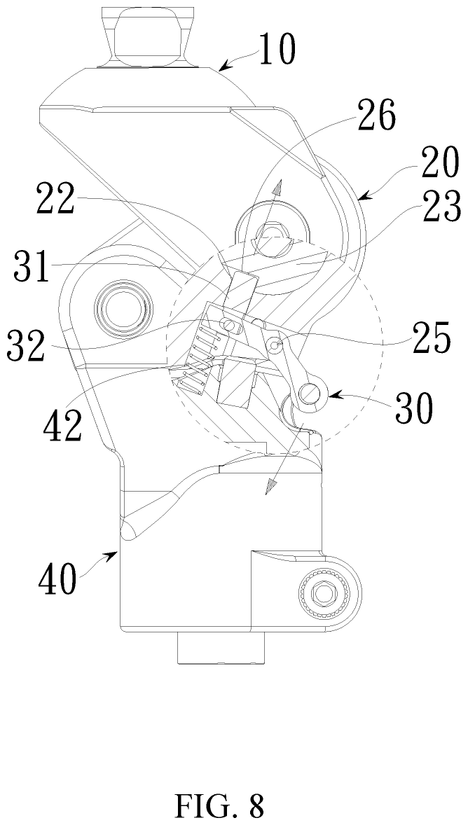

Further referring to FIGS. 7 and 8, as shown in the cross-section of B-B, when the pull cable (50) does not pull the connecting rod, the fixer (31) is moved upward in the fastening clamp channel (22) to reach into the axle opening (26) and becomes a retained condition, so as to set the knee joint in a fixed condition, where the knee upper seat (10) is not allowed to perform the rotating motion.

In summary, the improvement on a locking structure of a knee joint according to the present invention is such that a fastening clamp (20) is formed therein with a fastening clamp channel (22), and a fixer connection rod (30) that has one end connected to a fixer (31) is pivotally connected to a fastening clamp groove (24) provided in an outside of the fastening clamp (20), wherein a rotary axle (23) provided in the fastening clamp (20) is set in communication with the fastening clamp channel (22), so that when a pull cable (50) is pulled upward, from the outside, the fixer connection rod (30) is synchronously driven to cause the fixer (31) to move in the fastening clamp channel (22) to free the knee upper seat (10) to rotate without being subject to any constraint; and wherein when the pull cable (50) is released, the fixer (31) is set into engagement with an axle opening (26) provided in the outside of the rotary axle (23) to set the knee upper seat (10) in a constrained state. The present invention allows an artificial limb wearer to make switching between a fixed state and a rotatable state and may also effectively reduce an impact force and protect the structure from exposure to the outside in order to ensure security of use.

1. A knee joint, comprising: a joint upper seat, which is extended downward to form a pair of joint upper seat through-holes; a joint lower seat, which comprises an elastic body, the joint lower seat being extended upward to form a pair of joint lower seat through-holes; a fastening clamp, which is provided therein with a fastening clamp through-hole and a rotary axle, the rotary axle corresponding to and connected to the joint upper seat by a pair of mounting bolts, the fastening clamp through-hole corresponding to and connected to the joint lower seat through-holes by a connection shaft penetrating therethrough;
wherein a fastening clamp channel is formed in the fastening clamp and is extended to and in communication with the rotary axle, the rotary axle being formed with an axle opening, the fastening clamp being extended from an outside thereof to form a fastening clamp groove, the fastening clamp groove being collaborative with a positioning piece penetrating therethrough to pivotally connect a fixer connection rod in the fastening clamp groove, the fixer connection rod having an end, which is collaborative with an axle penetrating therethrough to penetrate through a fixer to set the fixer in the fastening clamp channel, the fixer connecting rod having an opposite end that is collaborative with a bolt to fix to a connecting rod, the connecting rod being coupled to a pull cable; wherein the connecting rod is pulled upward by the pull cable to rotate, the fixer moves downward in the fastening clamp channel to contact and push the elastic body so as to set the knee joint in a rotatable condition, and when the pull cable stops pulling the connecting rod, the fixer moves upward in the fastening clamp channel to reach into the axle opening to be set in a fixed condition, where the knee joint is set in a fixed condition,
wherein the axle opening is formed on a circumferential surface of the rotary axle and the fixer is movable in the fastening clamp channel along an axis line that intersects the circumferential surface, such that the fixer is movable between a first position where the fixer is located out of the axle opening and a second position where the fixer is located in the axle opening, and wherein in the rotatable condition of the knee joint, the fixer connection rod is at a first angular position to set the fixer at the first position and deform the elastic body such that relative rotation of the rotary axle with respect to the joint lower seat is enabled, and in the fixed condition of the knee joint, the fixer connection rod is at a second angular position and the elastic body is released to bias the fixer to the second position to get in engagement with the axle opening so as to prevent the relative rotation of the rotary axle with respect to the joint lower seat.

2. The knee joint according to claim 1, wherein the elastic body comprises a spring.
